(19) United States
(12) Patent Application Publication
(10) Pub. No.: US 2008/0086326 A1
(43) Pub. Date: Apr. 10, 2008

(54) SYSTEM AND APPARATUS FOR DISPENSING CONTROLLED PHARMACEUTICAL PRODUCTS

(76) Inventors: FERNANDO MOURA, Douglasville, PA (US); Dina Reese, Lancaster, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/539,717

(22) Filed: Oct. 9, 2006

(51) Int. Cl. G06Q 10/00; G06Q 20/00; G06Q 50/00; G06G 1/14

(52) U.S. Cl. 705/2; 705/22; 705/23

(57) ABSTRACT

The present invention is directed to a controlled dispensing system comprising at least one dispensing machine having a data input means for receiving information from an individual, an inventory of one or more products available for purchase provided that at least some of the products are scheduled products, a dispensing means for retrieving one or more products from the inventory and delivering such product(s) to a retrieval point, a verification means for establishing the identity of the individual; and a searchable database that contains information that correlates an individual's identity to purchases of at least one scheduled product, a method for use of such systems.

The present invention is directed to methods and systems to dispense pharmaceutical products in a controlled manner. This invention is an improved method for dispensing scheduled pharmaceutical products within the confines of a retail establishment.

### BACKGROUND

Vending machines are a well-known means for providing products to consumers without the need for a large physical store-front, significant retail space or constant presence of sales personnel. Vending machines can be located to maximize convenience for the consumer and directly managed on a periodic basis to check inventory, deposits, and general condition of the machine. Electronic vending machines have been developed that are capable of using paper currency, coins or credit cards to process payments. Vending machines capable of collecting and transmitting information that would required for sales estimates by a central computer system are known, as shown in U.S. Pat. No. 6,021,394. Vending machines have, in general, been used as a means to deliver products and process payments for selected products, not as a means to control and document the delivery of products.

The Combat Methamphetamine Epidemic Act of 2005 (Title VII of the USA PATRIOT Improvement and Reauthorization Act of 2005, P.L. 109-177) was signed into law Mar. 9, 2006. The CME Act primarily affected persons selling products containing ephedrine, pseudoephedrine, and phenylpropanolamine. Effective Mar. 9, 2006, the CME Act made products that contain ephedrine, pseudoephedrine, or phenylpropanolamine a “scheduled listed chemical product” though such products remain “non-prescription drugs”. Further rules came into effect on Apr. 8, 2006 that among other things set the daily sales limit of ephedrine base, pseudoephedrine base, or phenylpropanolamine base, regardless of the number of transactions. After Sep. 30, 2006, a regulated seller must place product containing these active ingredients such that customers do not have direct access before the sale is made (“behind the counter” placement) or in a locked cabinet that is located in an area of the facility to which customers do have direct access. A regulated seller must deliver product directly into the custody of the purchaser, though distributions of drug products by retail distributors that may not include face-to-face transactions are permitted to the extent such distributions are consistent with the activities authorized for a retail distributor as specified in Section 802(46) of the CME Act.

A seller of these scheduled products must maintain written or electronic list (logbook) of sales that identifies: (1) products by name; (2) quantity sold; (3) names and addresses of purchasers; and, (4) date and time of the sales. Additionally, a seller may not sell the scheduled product unless the prospective purchaser presents a photographic identification card issued by a state or the Federal Government, or a document otherwise considered acceptable by statute. A purchaser must sign the logbook and enter his or her name, address, and date and time of sale. The seller must determine that the name entered into the logbook corresponds to the name provided on such identification and that the date and time entered are correct. The seller must enter into the logbook the name of the product and the quantity sold. The logbook must contain a notice to prospective purchasers that entering false statements or misrepresentations in the logbook may subject the purchaser to criminal penalties under 18 U.S.C. § 1001 and such notice must specify the maximum fine ($250,000.00) and term of imprisonment (5 years). The seller must maintain each entry in the logbook for not fewer than two years after the date on which the entry is made.

This invention solves one or more problems facing retailers trying to comply with the dispensing and verification requirements of the CME Act. For the purposes of this invention the seller may be considered to be an individual such as a pharmacist or pharmacy technician, or an organization such as a retail pharmacy, or other retail organization. For example, under common circumstances, when the pharmacy is open, a customer must walk to a store shelf, identify the product that they intend to buy, pick a card of the desired product off the shelf, walk to the pharmacist, wait in line, present the “ticket” to the pharmacist or representative, and present a valid drivers' license or state approved ID card. The pharmacist then will first attempt to verify that the purchaser is on file, either via a computer database or a manual entry log, usually by checking the last name and confirm whether or not the customer is allowed to purchase the selected product. This review and verification step is relatively labor-intensive and a significant burden for all parties involved. This review and verification step also inherently has the potential for misrepresentation of identification on the part of the buyer. This step also has the potential for making mistakes in the documentation of the buyer's information on the part of the seller.

Another problem arises because many pharmacies are only open or staffed during certain hours of the day. Given the current review and verification procedures, a large segment of the population may not be able to purchase these scheduled products. This present invention allows a retailer to dispense these scheduled products in a controlled, documented and compliant manner.

Another problem can arise from the fact that logbooks cannot be easily cross-referenced with logbooks from other purchasing locations within a short period of time. This can allow for purchasers to go to multiple locations within a short period of time in order to buy scheduled products.

This invention solves the problem of the limited time available for pharmacists to check, register and dispense scheduled products. This invention also solves the problem of unused shelf space, which can be as many as 30-40 SKUs that are presently occupied by product “tickets” requiring further store inventory. This invention solves the foregoing problems and others by automating many requirements of the CME Act relating to the sale of scheduled products.

### SUMMARY OF THE INVENTION

The present inventions relate generally to methods and apparatus for dispensing scheduled pharmaceutical products to prospective purchasers in a controlled and documented manner. Initially, the customer approaches a dispensing unit containing one or more products that are scheduled or regulated in some fashion within the applicable jurisdiction. The customer selects one or more desired products from the inventory or menu of selections using a display or data entry unit. The display unit prompts the customer to input individual information using an approved method of identification, such as a driver's license, passport, credit card, or biometric measurement. A computer verifies that the customer has provided all of information required by statute or applicable regulations, including name, address, previous date of purchase (and amount) of a scheduled product, and age. The computer checks customer information against customer records retained in a searchable database and subsequently informs customer whether the purchase can be completed. If a purchase is permitted, the customer will be asked to complete the dispensing transaction by entering an electronic signature using a secure and authenticated means, such as electronic signature pad, password identification or biometric identification. The computer records the customer information and the amount of selected product dispensed electronically in the database or by providing a printout to the local pharmacist. The dispensing machine then provides the scheduled product to the customer. The customer pays for the dispensed product using cash, credit or debit payment at the customer counter in conventional manner. Alternatively, a vending transaction can be completed by the machine prior to dispensing the scheduled product using cash, credit, debit payments or by using a qualified account, such as a health saving account using a vending system described more fully in copending application MCP-5129USPNP (Ser. No. ______), which is incorporated herein by reference.

### BRIEF DESCRIPTION OF THE FIGURE

FIG. 1 is a schematic drawing of process steps for practicing one embodiment of the invention.

### DETAILED DESCRIPTION OF THE INVENTION

In particular, at a general level, the present invention is directed to a dispensing system and method of practice that has a means for inputting data, e.g., data input device, means for analyzing the data, e.g., computer, microprocessor and associated database(s), and an output based on the analysis that includes information, product, or both. In one embodiment, the apparatus only dispenses products and the purchaser is required to pay for the purchase by means other than through the dispensing system. By only dispensing product, such a system eliminates the requirements for a point of sale system, cash handling, change, receipt, out of paper issues, coupon handling etc. The only requirement for the retailer is to load inventory into racks or shelving. In an alternative embodiment, the apparatus is capable of both dispensing a scheduled product and completing a payment transaction for such product.

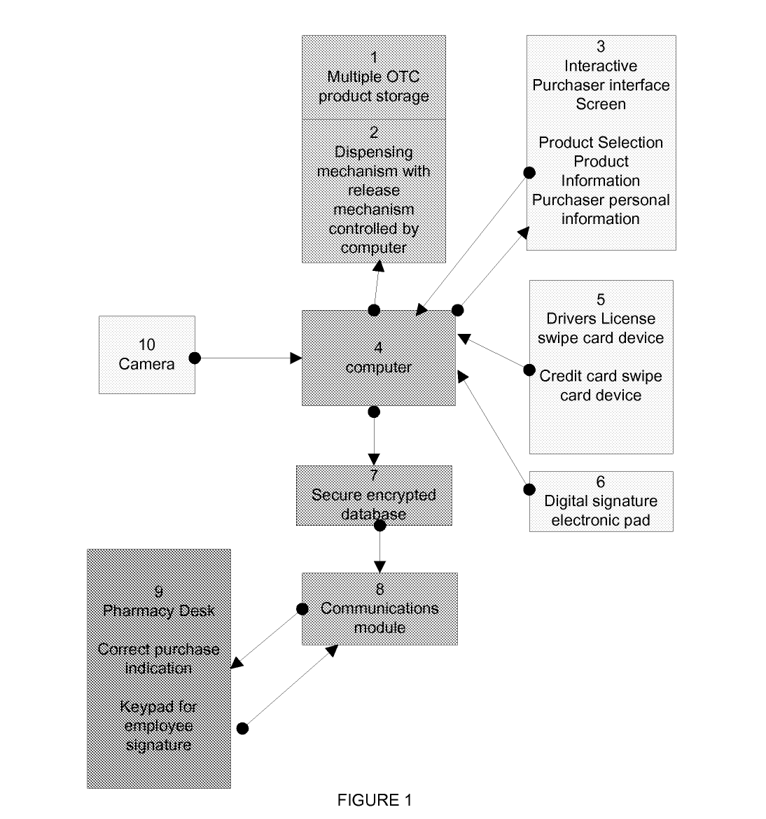

In one embodiment, the present invention includes at least ten (10) mechanical, electrical, electronic and software modules that are described more fully below and identified in the attached FIG. 1. The preferred dispensing machine is similar to a vending machine having shelving or racks that contain products, preferably healthcare products, such as pharmaceuticals, minerals, vitamins and other nutraceuticals, oral care agents and combinations thereof. Examples of healthcare products include but are not limited to analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, gastrointestinal agents, migraine preparations, motion sickness products, mucolytics, muscle relaxants, osteoporosis preparations, oral care agents, smoking cessation agents, polydimethylsiloxanes, respiratory agents, sleep-aids, urinary tract agents and mixtures thereof.

At least some of the healthcare products provided in the dispensing system are scheduled products, which are healthcare products that contain one or more active ingredients that are subject to regulatory requirements to control and document their distribution, a regulated active ingredient. Presently, one group of regulated active ingredients includes ephedrine, pseudoephedrine and phenylpropanolamine, including their salts, optical isomers, and salts of such optical isomers. Several states have also proposed including dextromethorphan on a list of regulated products. The healthcare products can contain pharmaceutically active ingredients in addition to the one or more active ingredients that are subject to regulatory requirements to control and document their distribution.

Examples of pharmaceutically active ingredients that can be combined with the foregoing regulated active ingredients, includes, but is not limited to: analgesics, anti-inflammatories, and antipyretics, e.g. non-steroidal anti-inflammatory drugs (NSAIDs), including a) propionic acid derivatives, e.g. ibuprofen, naproxen, ketoprofen and the like; b) acetic acid derivatives, e.g. indomethacin, diclofenac, sulindac, tolmetin, and the like; c) fenamic acid derivatives, e.g. mefenamic acid, meclofenamic acid, flufenamic acid, and the like; d) biphenylcarbodylic acid derivatives, e.g. diflunisal, flufenisal, and the like; e) oxicams, e.g. piroxicam, sudoxicam, isoxicam, meloxicam, and the like; f) cyclooxygenase-2 (COX-2) selective NSAIDs; and g) pharmaceutically acceptable salts of the foregoing. Examples of analgesics include acetaminophen, acetyl salicylic acid, ibuprofen, naproxen, ketoprofen, flurbiprofen, diclofenac, cyclobenzaprine, meloxicam, rofecoxib, celecoxib, and pharmaceutically acceptable salts, esters, isomers, and mixtures thereof. Still further, the additional pharmaceutically active ingredient can be selected from the group consisting of chlorpheniramine, dextromethorphan, diphenhydramine, astemizole, terfenadine, fexofenadine, loratadine, desloratadine, cetirizine, cemetidine, famotidine, omeprezole, cetirizine, mixtures thereof and pharmaceutically acceptable salts, esters, isomers, and mixtures thereof. Preferably, the scheduled product is available as a non-prescription product, notwithstanding the presence of at least one regulated active ingredient.

The invention consists of an assembly of integrated modules that form a unique product dispensing system. One component of the present invention is a product storage compartment that contains healthcare or personal care products. These products can be prescription or non-prescription pharmaceutical products, wherein at least some of the healthcare products contain a regulated active ingredient or the product itself is a scheduled product as described above.

Examples of healthcare products, include but are not limited to: analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, gastrointestinal agents, migraine preparations, motion sickness products, mucolytics, muscle relaxants, osteoporosis preparations, polydimethylsiloxanes, respiratory agents, sleep-aids, urinary tract agents and mixtures thereof. Healthcare products contemplated for use in the present systems can also encompass self-diagnostic devices, such as pregnancy tests, cholesterol tests, viral and infection tests, and blood glucose monitors and test strips and related consumables for such diagnostic devices.

The dispensing device must include a means for transferring one or more selected products from product inventory to a location from which individual can retrieve the selection. Examples such transfer means include robotic arms, conveyor belts, moving inventory racks that allow product to fall into a retrieval point and such other means known in the art.

The dispensing device can also include a data entry unit, such as a touch screen or keyboard and screen, wherein the data entry unit should be interactive. An interactive unit means that the individual is at least prompted by the device to enter required information and provide requisite forms of identification. It is possible that sequential instructions could be posted in lieu of an interactive data entry unit.

The dispensing device must include or be in communication with a computer, processor, network server and/or a searchable regulated database of information discussed below. The database can be based locally for purchases at the specific location and/or periodically updated to incorporate on sales of scheduled products from associated retail establishments. The dispensing machine, upon receiving required identification information and product selection, can then send the information either to a processing center for analysis or analyze the available information using an onboard computer and software and locally installed regulated database. The regulated database can be centrally located and maintained at that central location provided that a means for connecting with and communicating with the central location regulated database is available. The centrally located regulated database can be maintained by a retail entity, an association of retail entities, or government agency. The vending machine can communicate and connect with such a database or central monitoring system via a hard-wire connection, satellite transmission, local radio transmission, cellular networks, or Wi-Fi network connections or combinations thereof. The vending system could maintain a back-up system to utilize a locally stored database in the event the central database is unavailable for any reason.

One piece of information that is currently required for the purchase of a scheduled product is personal identification. There are many forms of personal identification, such as passports, birth certificates and driver's licenses. Increased requirements for security has caused many states to adopt new technologies to prevent fraud and enhance the ability to confirm personal identity using a driver's license. Hence, one means for providing requisite personal identification information could by having a swipe or scanner capable of reading such cards. Additionally, since one means for paying for a purchase is with a credit card, the dispensing system should preferably include a credit card scanner.

Prospective purchasers are statutorily required to review and authenticate the information provided by signature. A preferred means for securing the requisite signature is with a digital signature pad that is know in the art. Alternatively, prospective purchasers could affix electronic signatures by acknowledging and/or checking appropriate boxes on a touch screen display. The dispensing device can optionally allow the pharmacist to enter a countersignature or acknowledgment in similar fashion.

The dispensing device would have or be able to access and/or record in a secure searchable database having at least the following information: 1) customer name; 2) address; 3) date of birth; 4) a list of scheduled products that were purchased within a prescribed period of time; 6) the amount of regulated active and/or scheduled product dispensed in each such transaction; 7) sale date of such transaction; 8) an approved electronic signature of the prospective purchaser; 9) an approved electronic countersignature, if required. The database could also monitor sales information, such as units sold and out of stock product.

At some point in the transaction, a comparison must be made as against the personal information, the form of personal information, and the individual. One way to accomplish this step is for the cashier, pharmacist or other appropriately trained individual to perform this function prior to payment and departure. Alternatively, an automated comparison and verification could be done using a camera and face recognition software. A computer using commercially available face recognition software would compare a digitized picture from the driver's license against a real-timeimage of the individual purchaser. The dispensing device could then provide the purchaser with a receipt demonstrating identification authentication. In the event the face recognition program could not authenticate the individual's identity, then the individual could still proceed to the cashier, pharmacist or other appropriately trained individual for authentication. Upon authentication, the individual would complete the transaction by making payment if such payment had not already been made.

Another means of verifying personal identification is biometric analysis, where the device would have the ability to check a database based on a biometric measurement of the individual (i.e. retina scan, fingerprint scan, hand scan.) Another means of verifying personal identification includes photographic analysis, where the device would have the ability to take a photograph and check the photograph in a database.

In a further embodiment, vending system is equipped with means to verify the accuracy of the dispensed product, preferably prior to receipt by the prospective purchaser. One means for verifying accuracy is to include a bar code scanner at a point in the shelving where products are displayed and released for dispensing. Alternatively, the products in inventory can be equipped with RFID tags. Preferably, if the vending system detects an error, the release product will be diverted prior to delivery to the prospective purchaser and the electronic records will be corrected. These features will ensure that to the extent a purchaser is registered as purchasing a scheduled product, he or she actually receives such a product.

In practicing one embodiment of the present method, a prospective purchaser approaches a dispensing unit. The prospective purchaser selects one or more products using a display/data entry unit. The display prompts the prospective purchaser to enter individual data using an approved method of individual identification including a driver's license, passport, credit card, or biometric measurement. A computer reviews this information to ensure that the individual has provided or otherwise satisfies regulatory requirements, including providing the name, address, previous purchase date and amount, and age limitation. Next, the computer determines if the prospective purchaser is authorized to make the desired purchase using the secure searchable database. The prospective purchaser authenticates the previously entered information by providing their signature, preferably an electronic signature through an electronic signature pad. The prospective purchaser can also authenticate their identity using a biometric sensor. The computer records the customer information, the amount of product to be dispensed and time/date of the purchase in the searchable database or by providing a printout for the local pharmacist, who will enter the information manually in a logbook. The dispensing machine dispenses the requested product or products. The prospective purchase completes the transaction by making payment with either cash, credit or debit payment at the customer counter or through traditional vending transaction associated with the dispensing machine.

The information recorded in the database can be communicated with other network-connected dispensing machines in order to facilitate control of purchases at other locations. The dispensing device can generate a transaction report and forward the same electronically or as a hard copy report directly to the appropriate regulatory agencies such as the Food and Drug Administration (FDA), the Drug Enforcement Agency (DEA) or to appropriate state agencies.

In one embodiment, the system records a running logbook, which can be printed in order to capture information for any specific length of time (i.e. a single hour, day or week). Alternatively, the running logbook can be downloaded in electronic form onto any suitable means for storing electronic data such as a disk, flash drive, compact disc or external disk drive. The logbook can be used to capture such information as lot numbers of product dispensed, packaging types, quantities dispensed, and dosage form types (i.e. liquids, tablets, suspensions, coated tablets). The logbook settings can be altered to highlight purchases of different dosage form types. The logbook can also be set to highlight active ingredients, including products that may include a single active ingredient, and those that may include multiple active ingredient combinations, as products with multiple active ingredient combinations may be less desirable to those wishing to use a scheduled product for purposes other than those that are indicated.

In a still further embodiment, the invention provides a vending system and method for managing the treatment regimen of a human patient using an over-the-counter (OTC) medicine comprising providing to the patient an interactive therapy managing system for de-selecting the prospective consumer from the treatment regimen. In one embodiment, the vending system and method further comprises prompting the patient for information; receiving information from the patient, including information about said treatment regimen; comparing said information with disqualifying criteria for said medicine; and in the event the patient meets said disqualifying criteria, declining to dispense the selected medicine. A suitable interactive therapy system is disclosed in copending application Ser. No. 11/185,385, filed Jul. 20, 2005, which is incorporated herein by reference.

In one embodiment of the invention, the prospective purchaser is prompted for information. Information is received by the vending system, including information about the treatment regimen of the patient. The information received from the prospective purchaser is compared with the disqualifying criteria for the selected product, and in the event the prospective purchaser meets the disqualifying criteria, a recommendation is made to the prospective purchaser to modify the treatment regimen and/or the selected product is not dispensed to the prospective purchaser.

The information provided by the prospective patient may include a test result. The patient himself, a testing agent, or a physician may provide such test result to the vending system. In connection with this, the vending system may recommend alternatives to the patient about how and where to obtain a test result if no current results are available. Test results include, for example, bone density, blood pressure, and serum cholesterol levels. Among other things, the test result may indicate that the medication is ineffective, or has improved the condition, but not to the desired level, i.e., the test result is not within the desired target range after a specified duration of treatment. These results would result in the dispensing system not providing the selected product to the prospective purchaser.

In an alternative embodiment, the customer approaches the dispensing unit and selects one or more products using a display/data entry unit. The display unit prompts customer to produce a driver's license in known fashion to enable the transfer of personal information contained therein. A drivers' license module will read the 2D matrix or magnetic stripe on card and scan purchaser picture provided on the driver's license. A camera, such as a digital camera, scans an image of the prospective purchaser's face and compares it with the previously scanned image from the driver's license. Following authentication of identity, the computer checks information against the conditions and limits specified in the regulated database including name, address, age limitation (over 18). If the computer check is positive, the display unit prompts the prospective purchaser to enter credit card information or other means for payment. If customer is allowed to purchase, the display unit will prompt the customer to sign at the electronic signing pad. The pharmacist will receive request via local indication and enters store employee number at the local keypad. If face verification is allowed, the transaction may proceed without any further intervention. The machine will unlock product bin and dispense product. The customer then takes the selected product to a check-out counter and completes the transaction by making appropriate payment.

Machines designed to display and dispense products are known and commercially available. Consumers can interact with the vending system using a touch-screen and can make payments using a card swipe. Selected products can, for example, be retrieved from inventory with a robotic arm that delivers the selected product to a dispensing door. The dispensing machine is preferably centrally monitored, networked and controlled by a locally installed software system. Other vending machine designs could be utilized provided that appropriate means are available for communicating and networking with required processing systems described herein.

In one preferred embodiment, as shown in FIG. 1, the dispensing unit is comprised of multiple modules which include but are not limited to multiple OTC product storage (1) containers connected to a dispensing mechanism with a release mechanism (2) connected to and controlled by a computer (4); an interactive purchaser interface screen (3) that displays information including but not limited to product selection, product information, and purchaser personal information, connected and set to send and receive information to said computer; a swipe card device (5) for such personal identification cards such as a driver's license, credit card, or other card connected and set to send information to said computer for identification purposes; a digital signature electronic pad (6) connected and set to send information to said computer; a secure encrypted database (7) set to receive information from said computer wherein the database includes information such as but not limited to that in (3), (5) and (6); a communications module (8) set to receive information from said database; and a confirmation module set to send and receive information from communications module (9) wherein the confirmation and/or approval of a product purchase is made from a pharmacy desk, as an indication of a correct purchase or from a keypad set to confirm a purchase form an employee signature; a camera (10) connected and set to record a potential purchaser's picture and set to send said picture to said computer. For the purposes of this invention the term connection or “connected to” includes but is not limited to any means known in the art to send or receive information, such as any wired line, phone line, hard line, DSL line, cable line, or wireless connection.

I/We claim:

1. A controlled dispensing system comprising at least one dispensing machine having:
a. data input means for receiving information from an individual;
b. an inventory of one or more products available for purchase provided that at least some of the products are scheduled products;
c. a dispensing means for retrieving one or more products from the inventory and delivering such product(s) to a retrieval point;
d. verification means for establishing the identity of the individual; and
e. a searchable database that contains information that correlates an individual's identity to purchases of at least one scheduled product.

2. A controlled dispensing system according to claim 1 wherein the searchable database contains a compilation of required purchase information from more than one retail establishment.

3. A controlled dispensing system according to claim 1 wherein the products are personal care or healthcare products.

4. A controlled dispensing system according to claim 3 wherein at least some of the personal care or healthcare products are packaged in containers holding other than individual dose quantities.

5. A controlled dispensing system according to claim 1 wherein at least one of the healthcare products contains a regulated active ingredient.

6. A controlled dispensing system according to claim 1 wherein at least one of the healthcare products contains ephedrine, pseudoephedrine, propanolamine, or dextromethorphan.

7. A controlled dispensing system according to claim 1 wherein the verification means comprises a digital scanner for importing an image from an approved form of identification, a camera for importing a facial image of the individual and software for comparing the images.

8. A controlled dispensing system according to claim 1 wherein the verification means comprises a biometric sensor and an associated database containing unique physical characteristics of individual prospective purchasers.

9. A controlled dispensing system according to claim 1 wherein the system records logbook information comprising individual identity and product dispensed.

10. A controlled dispensing system comprising at least one dispensing machine having:
a. data input means for receiving information from an individual;
b. an inventory of one or more products available for purchase provided that at least some of the products are scheduled products;
c. a dispensing means for retrieving one or more products from the inventory and delivering such product(s) to a retrieval point; and
d. verification means for establishing the identity of the individual; and
e. a searchable database that contains information that correlates an individual's identity to purchases of at least one scheduled product;
wherein the dispensing machine is in communication with a central system that includes the searchable database.

11. A controlled dispensing system according to claim 10 wherein the searchable database contains a compilation of required purchase information from more than one retail establishment.

12. A controlled dispensing system according to claim 10 wherein the products are personal care or healthcare products.

13. A controlled dispensing system according to claim 1 wherein at least one of the healthcare products contains ephedrine, pseudoephedrine, propanolamine, or dextromethorphan

14. A controlled dispensing system according to claim 12 wherein at least some of the personal care or healthcare products are packaged in containers holding other than individual dose quantities.

15. A controlled dispensing system according to claim 10 wherein at least one of the healthcare products contains a regulated active ingredient.

16. A controlled dispensing system according to claim 10 wherein the verification means comprises a digital scanner for importing an image from an approved form of identification, a camera for importing a facial image of the individual and software for comparing the images.

17. A controlled dispensing system according to claim 10 wherein the verification means comprises a biometric sensor and an associated database containing unique physical characteristics of individual prospective purchasers.

18. A controlled dispensing system according to claim 10 wherein the system records logbook information comprising individual identity and product dispensed.

19. A method for selling scheduled products comprising providing at least one scheduled products to a prospective purchaser using a controlled dispensing system according to claim 1 in one or more retail establishments.

20. A method for selling scheduled products comprising providing to a prospective purchaser at least one scheduled product using a controlled dispensing system according to claim 10 in one or more retail establishments.
